(19) United States
(12) Plant Patent Application Publication
(10) Pub. No.: US 2012/0260381 P1
(43) Pub. Date: Oct. 11, 2012

(54) Heuchera plant named 'Vienna'

(50) ;

(75) Inventor: Janet N. Egger, Wilsonville, OR (US)

(73) Assignee: Terra Nova Nurseries, Inc.

(21) Appl. No.: 13/066,124

(22) Filed: Apr. 6, 2011

(51) Int. Cl. A01H 5/00

(52) U.S. Cl. PLT/440

(57) ABSTRACT

A new and distinct cultivar of Heuchera plant characterized by multicolored leaves with a heavy silver white veil, narrow wands of pink flowers, reblooming, and excellent tolerance to heat and humidity.

### BOTANICAL DENOMINATION

Heuchera spp.

### VARIETY DESIGNATION

‘Vienna’

### BACKGROUND OF THE INVENTION

The present invention relates to a new and distinct cultivar of Heuchera hybrid and given the cultivar name of ‘Vienna’. Heuchera is in the family Saxifragaceae. Heuchera ‘Vienna’ originated from a planned breeding cross between Heuchera 1021-1, a proprietary unreleased plant, as the seed parent, and Heuchera 1021-5, a proprietary unreleased plant, as the pollen parent. Compared to the seed parent the new cultivar has foliage colored rose red to khaki rather than yellow. Compared to the pollen parent the new cultivar has a pink rather than a white flower.

This new Heuchera is uniquely distinguished by:

1. multicolored leaves with a heavy silver white veil,
2. narrow wands of pink flowers,
3. reblooming,
4. excellent tolerance to heat and humidity.

This new cultivar has been reproduced only by asexual propagation (cuttings and tissue culture). Each of the progeny exhibits identical characteristics to the original plant. Asexual propagation by cuttings and tissue culture using standard micropropagation techniques with terminal and lateral shoots, as done in Canby, Oreg., shows that the foregoing characteristics and distinctions come true to form and are established and transmitted through succeeding propagations. The present invention has not been evaluated under all possible environmental conditions. The phenotype may change with variations in environment without a change in the genotype of the plant.

### BRIEF DESCRIPTION OF THE DRAWINGS

FIG. 1 shows a one-year-old plant of Heuchera ‘Vienna’ growing in a one gallon container in greenhouse in June in Canby, Oreg.

FIG. 2 shows a one-year-old grown in the trial field in full sun in September in Canby, Oreg.

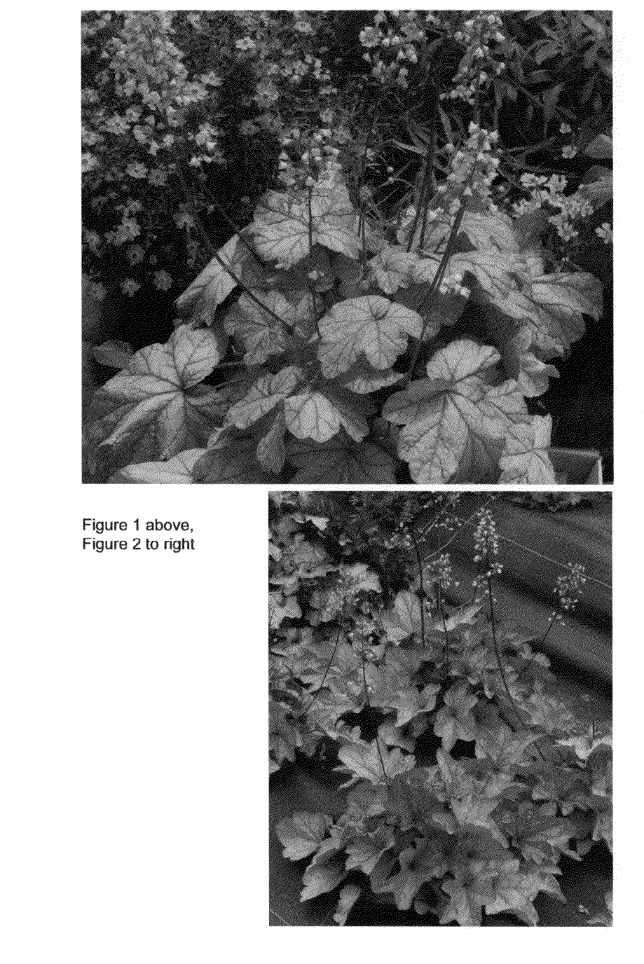

### DETAILED PLANT DESCRIPTION

The following is a detailed description of the new Heuchera cultivar based on observations taken in September of a one-year-old specimen grown in a one gallon container in a shade house in Canby, Oreg. Canby is Zone 8 on the USDA Hardiness map. Temperatures range from a high of 95 degrees F. in August to an average of 32 degrees F. in January. Normal rainfall in Canby is 42.8 inches per year. The color descriptions are all based on The Royal Horticultural Society Colour Chart, 5th edition.
Plant:
   Form.—basal rosette, herbaceous perennial.
   Hardiness.—USDA Zone 4 to 9.
   Size.—13 cm tall from top of the foliage to the ground and 36 cm wide.
   Habit.—mounding.
   Crown number.—7.
   Vigor.—good.
   Roots.—fibrous, freely branching, fine, and white in color; roots develop easily from cuttings.
Leaf:
   Type.—simple.
   Arrangement.—rosette.
   Shape.—broadly ovate.
   Lobing/division.—5 to 9 shallow lobes.
   Venation.—palmate.
   Margins.—crenate.
   Apex.—mucronulate.
   Base.—cordate.
   Blade size.—grows to 13 cm long and 12 cm wide.
   Surface texture.—glandular on both sides.
   Petiole description.—grows to 12 cm long and 3 mm wide, glandular hairs, Greyed Purple 187C.
   Leaf color.—variable depending amount of light, temperature, and age of the leaf; new growth topside Greyed Red 178A on veins and margins but covered over with a veil of silver white, closest to Black 202D, giving a blended color; bottom side Red Purple 59A; mature leaves lighten to Grey Brown N199B with veil Greyed Green 198A; older leaves are Green N137A with veil Greyed Green 198A.
Inflorescence:
   Size.—grows to 18 cm long and 7 cm wide.
   Type.—thyrse.
   Number of flowers per thyrse.—about 100 per thyrse.
   Number of thyrse.—about 10 in first bloom.
   Peduncle.—grows to 36 cm tall, 4 mm wide at base, Greyed Purple 187A, glandular hairs, with no stem leaves.
   Pedicel.—variable in size, with glandular hairs, Greyed Purple 187A.
   Bloom period.—July to October in Canby, Oreg.
Flower bud:
   Size.—3 mm wide and 6 mm long.
   Description.—glandular puberulent, ovoid.
   Color.—Red 55C on top ⅔ and Red 55A on bottom ⅓.
Flower:
   Type.—perfect.
   Shape.—campanulate.
   Size.—6 mm long and 6 mm wide.
   Petal description.—5 in number, 3 mm long and 1 mm wide, reflexed, spatulate, tip obtuse, base attenuate, margin entire to irregular, sparsely glandular on both sides; Red 55D with Red 55B on tip.
   Calyx description.—campanulate, 6 mm long and 6 mm wide, with 5 lobes, divided ⅔ way to the base, each 3 mm long and 2.7 mm wide, glandular hairs on both sides, tip obtuse, margin entire, outside color Red Purple 58A on bottom to Red 55B to Red 55D on lobes to Red 53A on lobe tips, inside Red 55D with tips Red 53A.
   Stamen description.—5 in number, filaments 2 mm long, White N155B, anthers 0.5 mm, Red Orange 32C, pollen none, male sterile.
   Pistil description.—1, 2-beaked, 4 mm long, ovary 2 mm long and Yellow White158B, style 2 mm long and White NN155B.
   Fragrance.—none.
   Lastingness.—each thyrse blooms for about 2 weeks on the plant.
Fruit and seed: none produced
Pest and disease tolerance: Excellent disease resistance to powdery mildew, the common problem of Heuchera. Susceptible to Heuchera rust and root weevils.

I claim:

1. A new and distinct cultivar of Heuchera plant as herein illustrated and described.
